(19) United States
(12) Patent Application Publication
(10) Pub. No.: US 2024/0410758 A1
(43) Pub. Date: Dec. 12, 2024

(54) CORE BODY TEMPERATURE ESTIMATION DEVICE, CORE BODY TEMPERATURE ESTIMATION METHOD, AND RECORDING MEDIUM

(71) Applicant: Panasonic Intellectual Property Management Co., Ltd., Osaka (JP)

(72) Inventor: Kenichiro NOSAKA, Osaka (JP)

(21) Appl. No.: 18/697,965

(22) PCT Filed: Sep. 21, 2022
(86) PCT No.: PCT/JP2022/035123

§ 371 Date: Apr. 2, 2024

(30) Foreign Application Priority Data
Oct. 19, 2021 (JP) 2021-171170

(51) Int. Cl. G01J 5/00; A61B 5/01; G06T 7/143

(52) U.S. Cl. CPC G01J 5/0025; A61B 5/01; G06T 7/143; G01J 2005/0077; G06T 2207/30201

(57) ABSTRACT

A core body temperature estimation method includes a thermal image obtaining step obtaining a target thermal image, a probability image generating step generating a region probability image showing a probability of each of a plurality of pixels in the target thermal image corresponding to a face part of a person, a region dividing step dividing the target thermal image into face part regions, a region generating step generating an estimation target region, a resolving power conversion step converting a partial thermal image corresponding to the estimation target region to a predetermined resolving power and a partial region probability image corresponding to the estimation target region (Sq) to the predetermined resolving power, and an output step of outputting an estimated value of a core body temperature by inputting the partial thermal image and the partial region probability image converted to the predetermined resolving power into a trained body temperature estimation model.

### CROSS-REFERENCE OF RELATED APPLICATIONS

This application is the U.S. National Phase under 35 U.S.C. § 371 of International Patent Application No. PCT/JP2022/035123, filed on Sep. 21, 2022, which in turn claims the benefit of Japanese Patent Application No. 2021-171170, filed on Oct. 19, 2021, the entire disclosures of which applications are incorporated by reference herein.

### TECHNICAL FIELD

The present disclosure relates to a core body temperature estimation device, a core body temperature estimation method, and a recording medium for estimating a person's core body temperature.

### BACKGROUND ART

There is known to date a temperature measuring device that measures a person's body temperature contactlessly (see, for example, Patent Literature (PTL) 1).

### CITATION LIST

### Patent Literature

### [PTL 1]

Japanese Unexamined Patent Application Publication No. 2021-139706

### SUMMARY OF INVENTION

### Technical Problem

Measurement results obtained by temperature measuring devices such as the one disclosed in PTL 1 above, however, are often susceptible to fluctuation depending on the environment where the temperature is measured or on the body part of which the temperature is measured. Thus, in terms of the accuracy, such a temperature measuring device may not be able to measure (i.e., output) a person's body temperature appropriately. Accordingly, the present disclosure provides a core body temperature estimation method and so forth that make it possible to output a person's body temperature more appropriately.

### Solution to Problem

To solve the problem above, a core body temperature estimation method according to one aspect of the present disclosure is a core body temperature estimation method of estimating a core body temperature of a person, and the core body temperature estimation method includes: obtaining a target thermal image that shows a radiation temperature distribution of a target space including a face of the person; generating a region probability image that shows a probability of each of a plurality of pixels in the target thermal image obtained corresponding to each of one or more face parts of the person; dividing the target thermal image into one or more face part regions corresponding to the one or more face parts, based on the region probability image generated; generating an estimation target region that includes, among the one or more face part regions, a region to be used to estimate the core body temperature of the person; converting, of the target thermal image, a partial thermal image corresponding to the estimation target region generated to an image of a predetermined resolving power and converting, of the region probability image, a partial region probability image corresponding to the estimation target region generated to the predetermined resolving power; and outputting an estimated value of the core body temperature of the person by inputting the partial thermal image converted to the predetermined resolving power and the partial region probability image converted to the predetermined resolving power into a body temperature estimation model trained and by causing the trained body temperature estimation model to calculate the estimated value of the core body temperature of the person.

Meanwhile, a recording medium according to one aspect of the present disclosure is a non-transitory computer-readable recording medium having recorded thereon a program for causing a computer to execute the core body temperature estimation method described above.

Meanwhile, a core body temperature estimation device according to one aspect of the present disclosure is a core body temperature estimation device that estimates a core body temperature of a person, and the core body temperature estimation device includes: a thermal image obtainer that obtains a target thermal image that shows a radiation temperature distribution of a target space including a face of the person; a probability image generator that generates a region probability image that shows a probability of each of a plurality of pixels in the target thermal image obtained corresponding to each of one or more face parts of the person; a region divider that divides the target thermal image into one or more face part regions corresponding to the one or more face parts, based on the region probability image generated; a region generator that generates an estimation target region that includes, among the one or more face part regions, a region to be used to estimate the core body temperature of the person; a resolving power converter that converts, of the target thermal image, a partial thermal image corresponding to the estimation target region generated to a predetermined resolving power and converting, of the region probability image, a partial region probability image corresponding to the estimation target region generated to the predetermined resolving power; and an outputter that outputs an estimated value of the core body temperature of the person by inputting the partial thermal image converted to the predetermined resolving power and the partial region probability image converted to the predetermined resolving power into a trained body temperature estimation model and by causing the trained body temperature estimation model to calculate the estimated value of the core body temperature of the person.

### Advantageous Effects of Invention

The present disclosure makes it possible to output a person's body temperature more appropriately.

### BRIEF DESCRIPTION OF DRAWINGS

FIG. 1 is a schematic diagram for describing a use case example of a core body temperature estimation device according to an embodiment.

FIG. 2 is a block diagram showing a functional configuration of a core body temperature estimation device according to an embodiment.

FIG. 3 is a diagram for describing an operation of a region generator according to an embodiment.

FIG. 4 is a diagram for describing an operation of an outputter according to an embodiment.

FIG. 5 is a diagram for describing an operation of an outputter according to a different example of an embodiment.

FIG. 6 is a diagram for describing an operation of an outputter according to yet another different example of an embodiment.

FIG. 7 is a flowchart showing an operation of a core body temperature estimation device according to an embodiment.

FIG. 8 is a first diagram for describing each image generated as a core body temperature estimation device according to an embodiment operates.

FIG. 9 is a second diagram for describing each image generated as a core body temperature estimation device according to an embodiment operates.

FIG. 10 is a graph showing relationships between estimated values and correct values of core body temperatures according to an example.

FIG. 11 is a graph showing relationships between measured values and correct values of body temperatures measured by a body temperature measuring device according to a comparative example.

### DESCRIPTION OF EMBODIMENTS

### Overview of the Present Disclosure

There is known to date a radiation thermometer that serves as a body temperature measuring device, and such a body temperature measuring device measures a person's body temperature by receiving radiation light emitted from that person (see, for example, PTL 1). Basically, such a radiation thermometer can measure merely the surface temperature of a body. The surface temperature of a body is known to be readily affected by, for example, the outside temperature and to fluctuate easily depending on the situation. Therefore, body temperature measuring devices of this type have shortcomings in that their measurement results show a low degree of correlation with core body temperatures, or the true body temperatures, and in that they cannot measure (i.e., output) a person's body temperature appropriately in terms of the accuracy.

A core body temperature estimation device according to the present disclosure inputs a thermal image that includes a target person into a trained model that has been trained in advance through machine learning, and causes the trained model to calculate a core body temperature through estimation, and outputs an estimated value of the core body temperature. Estimating a core body temperature in this manner makes it possible to obtain an estimated value of the body temperature that is closer to its true value. In other words, such a technique makes it possible to output a person's body temperature more appropriately. The core body temperature estimation device according to the present disclosure uses a thermal image alone and thus has the following advantages.

While a thermal image is an image that includes a target person, such an image is less likely to include personal information that allows the target person to be distinguished from others. Typically, in order to estimate a core body temperature, it is desirable that a person's body part whose temperature is likely to correlate with the core body temperature be identified and the radiation temperature (i.e., a thermal image) of that body part alone be used. In other words, a visible light image or the like needs to be used in combination in order to identify body parts of the target person. However, such a visible light image is likely to include personal information, and there may be a large number of people who feel uncomfortable with their visible light images containing personal information being used in this instance. In this respect, if a core body temperature can be estimated from a thermal image alone, such a technique offers advantages in terms of protecting privacy of a person subjected to the measurement. Furthermore, even in a case in which a visible light image is used as described above, a thermal image has to be used as well. Therefore, if a core body temperature can be estimated with the use of a thermal image alone, that renders unnecessary any configuration for obtaining a visible light image, and this offers advantages in terms of the device cost.

A core body temperature estimation method according to a first aspect of the present disclosure is, specifically, as follows.

The core body temperature estimation method is a core body temperature estimation method of estimating a core body temperature of a person, and the core body temperature estimation method includes obtaining a target thermal image that shows a radiation temperature distribution of a target space including a face of the person; generating a region probability image that shows a probability of each of a plurality of pixels in the target thermal image obtained corresponding to each of one or more face parts of the person; dividing the target thermal image into one or more face part regions corresponding to the one or more face parts, based on the region probability image generated; generating an estimation target region that includes, among the one or more face part regions, a region to be used to estimate the core body temperature of the person; converting, of the target thermal image, a partial thermal image corresponding to the estimation target region generated to an image of a predetermined resolving power and converting, of the region probability image, a partial region probability image corresponding to the estimation target region generated to the predetermined resolving power; and outputting an estimated value of the core body temperature of the person by inputting the partial thermal image converted to the predetermined resolving power and the partial region probability image converted to the predetermined resolving power into a trained body temperature estimation model and by causing the trained body temperature estimation model to calculate the estimated value of the core body temperature of the person.

With such a core body temperature estimation method, a target thermal image of a person can be obtained, and the core body temperature of this person can be estimated from the obtained target thermal image. In order to estimate the core body temperature, the core body temperature estimation method uses the target thermal image and the region probability image that shows the probability of each of the pixels in the target thermal image corresponding to each of the one or more face parts of the person. Therefore, the core body temperature estimated herein is output based on the estimation that takes into consideration the information about the temperature value at each pixel in the thermal image and the information indicating to which of the one or more parts of the person's face each pixel may correspond. Furthermore, the estimated value of the core body temperature to be estimated is output as the items of information above are input to the trained body temperature estimation model in advance through machine learning. In the body temperature estimation model, a relatively large weighting factor is assigned to, among the temperature values of the pixels in the thermal image, a temperature value of the pixel with a high probability that the pixel corresponds to the face part that has a high correlation with the true core body temperature value, and thus a more accurate core body temperature can be estimated and output. In particular, in the core body temperature estimation method, the target thermal image alone is used, and thus as compared to an example in which a thermal image is combined with a visible light image, the estimated value of the core body temperature can be output more appropriately from the standpoint of the privacy protection or the device cost.

A core body temperature estimation method according to a second aspect of the present disclosure is the core body temperature estimation method according to the first aspect in which, in the converting, an input image is converted to an image of the predetermined resolving power through at least one of a process of reducing a size of the input image or a process of interpolating the input image.

According to this aspect, the resolving power of the partial thermal image corresponding to the generated estimation target region or of the partial region probability image corresponding to the generated estimation target region can be converted by adopting at least one of the process of reducing the image or the process of interpolating the image.

A core body temperature estimation method according to a third aspect of the present disclosure is the core body temperature estimation method according to the first or second aspect in which the one or more face parts include at least one of a part that has on a mask serving as an accessory or a part that has on eyeglasses serving as an accessory.

According to this aspect, a part having a mask on and a part having eyeglasses on can be identified as one of the face parts, and thus a person's more accurate core body temperature can be estimated and output even for a person having a mask on or a person having eyeglasses on.

A core body temperature estimation method according to a fourth aspect of the present disclosure is the core body temperature estimation method according to the third aspect in which, in the outputting, wearing state information that causes a display device to display a wearing state is output, the wearing state indicating whether the mask is worn.

According to this aspect, the wearing state indicating whether a mask is worn can be displayed on the display device with the use of the wearing state information.

A core body temperature estimation method according to a fifth aspect of the present disclosure is the core body temperature estimation method according to any one of the first to fourth aspects that further includes determining whether a thermal image includes the face of the person or does not include the face of the person, and in the obtaining, a thermal image determined to include the face of the person is obtained as the target thermal image.

According to this aspect, as only a thermal image that includes the face of the person is obtained as the target thermal image, for example, any thermal images that do not include the face of the person can be kept from being obtained as the target thermal image. In other words, the process of estimating the core body temperature can be kept from being performed on a thermal image that does not include the face of the person, and thus the above aspect is useful in terms of saving processing resources.

A core body temperature estimation method according to a sixth aspect of the present disclosure is the core body temperature estimation method according to any one of the first to fifth aspects in which, in the generating of the estimation target region, a center of gravity coordinate of a face part region is calculated, the face part region corresponding to one or more parts, among the one or more face parts, excluding a part above eye parts of the person and a part below a chin part of the person, and a region having, as a center, the center of gravity coordinate calculated is generated as the estimation target region.

According to this aspect, the estimation target region can be generated with the face parts set to the parts excluding any parts above the eye parts of the person and any parts below the chin part of the person. The estimation target region is preferably of the same size as much as possible across two or more target thermal images obtained at different timings while each estimation target region includes a large portion (e.g., the entirety) of the face of the person. However, the area is not stable in the above excluded parts because of the hair or the clothing, and thus these parts are not suitable for determining the center of the estimation target region. In other words, as the center of the estimation target region is determined with such regions excluded, the size of the estimation target region can be stabilized.

A core body temperature estimation method according to a seventh aspect of the present disclosure is the core body temperature estimation method according to any one of the first to sixth aspects in which, in the outputting, core body temperature information that causes a display device to display the estimated value of the core body temperature is output.

According to this aspect, the estimated value of the core body temperature estimated can be displayed on the display device with the use of the core body temperature information.

A core body temperature estimation method according to an eighth aspect of the present disclosure is the core body temperature estimation method according to any one of the first to seventh aspects in which, in the outputting, warning information for issuing a warning is output when the estimated value of the core body temperature is higher than or equal to a predetermined value.

According to this aspect, with the use of the warning information, a warning can be issued based on the fact that the estimated value of the core body temperature estimated is higher than or equal to the predetermined value.

A core body temperature estimation method according to a ninth aspect of the present disclosure is the core body temperature estimation method according to any one of the first to eighth aspects in which the outputting includes a process of calculating a mean value of all pixels in an image corresponding to the partial thermal image converted to the predetermined resolving power and the partial region probability image converted to the predetermined resolving power.

According to this aspect, the estimated value of the core body temperature of the person can be output based on the mean value of all the pixels in the image corresponding to the partial thermal image converted to the predetermined resolving power and the image corresponding to the partial region probability image converted to the predetermined resolving power.

A recording medium according to a tenth aspect of the present disclosure is a non-transitory computer-readable recording medium having recorded thereon a program that causes a computer to execute the core body temperature estimation method according to any one of the first and ninth aspects above.

According to this aspect, advantageous effects similar to those provided by the core body temperature estimation method described above can be obtained with the use of a computer.

A core body temperature estimation device according to an eleventh aspect of the present disclosure is a core body temperature estimation device that estimates a core body temperature of a person, and the core body temperature estimation device includes a thermal image obtainer that obtains a target thermal image that shows a radiation temperature distribution of a target space including a face of the person; a probability image generator that generates a region probability image that shows a probability of each of a plurality of pixels in the target thermal image obtained corresponding to each of one or more face parts of the person; a region divider that divides the target thermal image into one or more face part regions corresponding to the one or more face parts, based on the region probability image generated; a region generator that generates an estimation target region that includes, among the one or more face part regions, a region to be used to estimate the core body temperature of the person; a resolving power converter that converts, of the target thermal image, a partial thermal image corresponding to the estimation target region generated to a predetermined resolving power and converting, of the region probability image, a partial region probability image corresponding to the estimation target region generated to the predetermined resolving power; and an outputter that outputs an estimated value of the core body temperature of the person by inputting the partial thermal image converted to the predetermined resolving power and the partial region probability image converted to the predetermined resolving power into a trained body temperature estimation model and by causing the trained body temperature estimation model to calculate the estimated value of the core body temperature of the person.

Such a core body temperature estimation device can provide advantageous effects similar to those provided by the core body temperature estimation method described above.

A core body temperature estimation device according to a twelfth aspect of the present disclosure is the core body temperature estimation device according to the eleventh aspect in which the resolving power converter converts an input image to an image of the predetermined resolving power through at least one of a process of reducing a size of the input image or a process of interpolating the input image.

According to this aspect, the resolving power of the partial thermal image corresponding to the generated estimation target region or of the partial region probability image corresponding to the generated estimation target region can be converted by adopting at least one of the process of reducing the image or the process of interpolating the image.

Hereinafter, an embodiment of the present disclosure will be described with reference to the drawings. The embodiment described below merely illustrates general or specific examples of the present disclosure. As such, the numerical values, the constituent elements, the arrangement and the connection modes of the constituent elements, the steps, the order of the steps, and so forth illustrated in the following embodiment are examples and are not intended to limit the present disclosure. Accordingly, among the constituent elements in the following embodiment, any constituent elements that are not cited in the independent claims each expressing the broadest concept of the present disclosure will be construed as optional constituent elements.

Moreover, the drawings are schematic diagrams and do not necessarily provide exact depictions. Therefore, the scales and so on do not necessarily match among the drawings. In the drawings, substantially identical components are given identical reference characters, and duplicate description thereof will be omitted or simplified.

### Embodiment

### Configuration

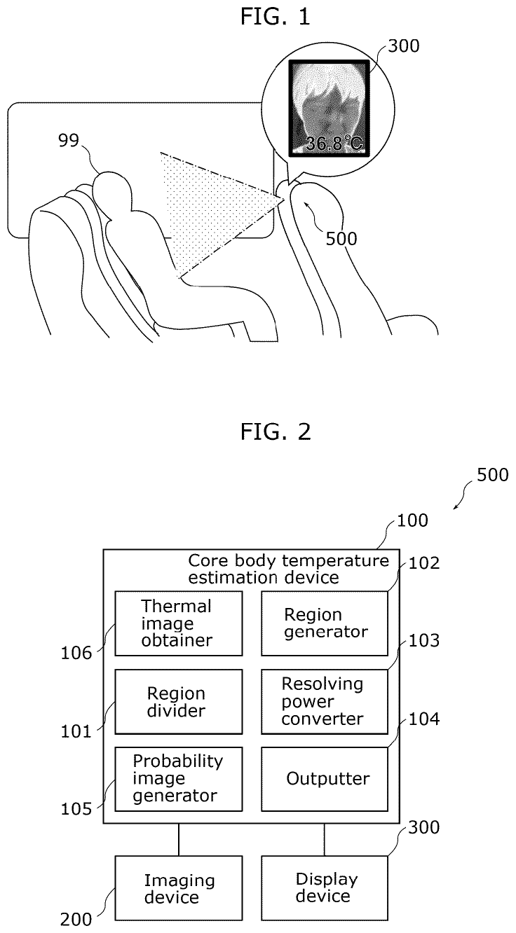

First, a configuration of a core body temperature estimation device according to an embodiment will be described with reference to FIG. 1 and FIG. 2. FIG. 1 is a schematic diagram for describing a use case example of the core body temperature estimation device according to the embodiment. Meanwhile, FIG. 2 is a block diagram showing a functional configuration of the core body temperature estimation device according to the embodiment.

As shown in FIG. 1, core body temperature estimation device 100 (see FIG. 2) according to the present embodiment is a device that, for example, obtains a thermal image capturing target person 99 (also referred to below simply as person 99) and outputs a core body temperature of person 99. Core body temperature estimation device 100 is embedded, for example, in core body temperature estimation system 500. Core body temperature estimation system 500 includes, in addition to core body temperature estimation device 100, imaging device 200 (see FIG. 2) for capturing a thermal image and display device 300 for displaying an output estimated value of a core body temperature. Core body temperature estimation system 500 is installed in, for example but not limited to, seats of public transportation facilities expected to be used by large numbers of general public, and estimates a core body temperature of person 99 who uses such seats. Thus, core body temperature estimation system 500 can promptly detect, from among the users using a public transportation facility, person 99 with an abnormal core body temperature. Examples of states possibly indicating an abnormal core body temperature include a state of low body temperature (predetermined value: 35.0° C. or lower) in which the core body temperature drops abruptly or a state of elevated body temperature (predetermined value: 37.0° C. or higher, 37.5° C. or higher, or 38.0° C. or higher) in which the core body temperature is abnormally high.

In core body temperature estimation system 500, imaging device 200 is disposed in such an orientation that allows imaging device 200 to capture an image of, in particular, the face of person 99 (e.g., in such an orientation that allows imaging device 200 to capture an image of the dot-hatched region within the angle of view defined by the dashed-dotted lines in FIG. 1). Core body temperature estimation system 500 captures a thermal image and transmits the captured thermal image to core body temperature estimation device 100. Core body temperature estimation device 100 receives the thermal image. Core body temperature estimation device 100 then calculates an estimated value of the core body temperature of person 99 that can be estimated from the received thermal image and outputs the calculated estimated value. The output estimated value is displayed on, for example but not limited to, display device 300. Display device 300 may display, along with the estimated value of the core body temperature, a part of the captured thermal image. With such a configuration, when looking at display device 300, person 99 can be informed that his or her thermal image has been used to estimate the core body temperature. Furthermore, displaying the thermal image can implicitly let person 99 know that no image that may include a relatively large amount of personal information, such as a visible light image, has been obtained, and thus this configuration can reduce any discomfort that person 99 might feel if his or her personal information is obtained.

As shown in FIG. 2, core body temperature estimation device 100 includes thermal image obtainer 106, region divider 101, probability image generator 105, region generator 102, resolving power converter 103, and outputter 104.

Thermal image obtainer 106 is a functional unit that obtains a target thermal image showing a radiation temperature distribution of a target space that includes the face of person 99. Thermal image obtainer 106 receives a thermal image that imaging device 200 outputs upon capturing an image of a space within the region defined by its angle of view, and obtains this thermal image as a target thermal image. While detailed description will be given later, if thermal image obtainer 106 receives, of thermals images that imaging device 200 outputs upon capturing an image of a space within the region defined by its angle of view, a thermal image that does not include person 99 (a space thermal image), thermal image obtainer 106 refrains from obtaining such a thermal image. This configuration, as a result, keeps core body temperature estimation device 100 from starting to operate, and the configuration is thus useful in terms of the energy cost. In the following description, unless otherwise indicated in particular, the terms “thermal image” and “target thermal image” are used synonymously to mean a target thermal image that includes the face of person 99.

Thermal image obtainer 106 is implemented as a computer including a processor and a memory executes a program set in advance. Likewise, region divider 101, probability image generator 105, region generator 102, resolving power converter 103, and outputter 104 described below are implemented as respective programs set therefor are executed. In other words, core body temperature estimation device 100 is implemented by a processor, a memory, and respective programs corresponding to thermal image obtainer 106 described above, region divider 101, probability image generator 105, region generator 102, resolving power converter 103, and outputter 104.

Probability image generator 105 generates a region probability image indicating the probability that each of a plurality of pixels in a thermal image corresponds to each of one or more face parts of person 99. The region probability image includes the probability that each of the pixels corresponds to a first part among the one or more face parts, the probability that each of the pixels corresponds to a second part among the one or more face parts, . . . , and the probability that each of the pixels corresponds to an nth part among the one or more face parts. In other words, the region probability image can be regarded as one image in which each pixel has n channels (pixel values) or as a set of n images including an image indicating the distribution of degrees of likeness of each pixel to a first part, an image indicating the distribution of degrees of likeness of each pixel to a second part, . . . , and an image indicating the distribution of degrees of likeness of each pixel to an nth part. According to the present embodiment, face parts include, in addition to the parts, such as the forehead, the eyes, the nose, the cheeks, the mouth, and the chin, that are actually present in the face of person 99, parts covered with accessories, such as a mask or eyeglasses. Therefore, even when person 99 has such accessories on, core body temperature estimation device 100 can estimate the core body temperature appropriately. Herein, face parts may further include a background portion of an image where person 99 is not included in the image.

Based on the generated region probability image, region divider 101 divides the thermal image into one or more face part regions each corresponding to one of the one or more face parts. Region divider 101 classifies each of the entire pixels in the region probability image into any one of the one or more face parts by assigning each of the pixels to the face part with the highest probability. Region divider 101 then determines the set of pixels classified into the same face part as one face part region. Each of the one or more face part regions is linked to the information (address information or the like) of the pixels classified to the corresponding face part region.

Probability image generator 105 and region divider 101 are implemented through a semantic segmentation model. The semantic segmentation model to be employed herein may be any existing semantic segmentation models, and examples of such include, but are not limited to, U-Net, DeepLab, and MaskFormer.

Region generator 102 generates an estimation target region that includes, from among the divided one or more face part regions, a region or regions to be used to estimate the core body temperature of person 99. In other words, region generator 102 reduces the influence of any face part regions that are not suitable for use in estimating the core body temperature, by removing such face part regions that are not suitable for use in estimating the core body temperature.

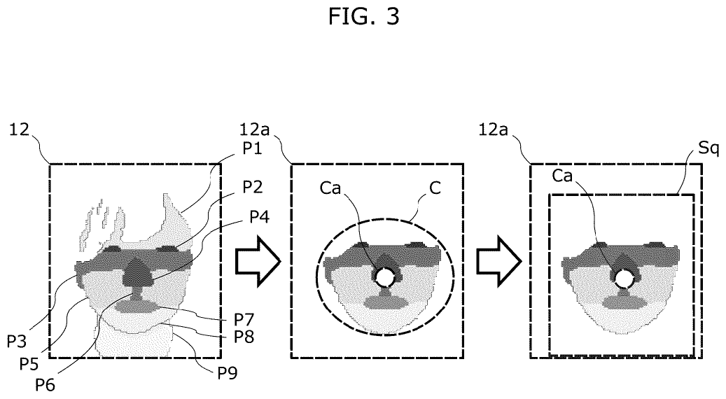

FIG. 3 is a diagram for describing an operation of the region generator according to the embodiment. FIG. 3 shows face part region divided image 12 indicating the distribution of the divided face part regions that include first part P1, second part P2, third part P3, fourth part P4, fifth part P5, sixth part P6, seventh part P7, eighth part P8, and ninth part P9, as well as the process of obtaining estimation target region Sq generated from face part region divided image 12. The size of estimation target region Sq is preferably stable (consistent) as much as possible across different thermal images, so that the size of an image input to a body temperature estimation model later is stabilized. Meanwhile, it is desirable that, in the estimation of a core body temperature, estimation target region Sq be set to a region that includes the entire face of person 99 and that can keep from being included any background portions that may have a negative influence on the estimation of the core body temperature of person 99. Therefore, estimation target region Sq generated below is generated through a size stabilization process of keeping the size of estimation target region Sq from greatly varying among thermal images and generated so as to have a size that includes the entire face of person 99.

In face part region divided image 12 shown in FIG. 3, first part P1 corresponds to the forehead, second part P2 corresponds to the eyes, eighth part P8 corresponds to the chin, and ninth part P9 corresponds to the neck. First, the center coordinate of estimation target region Sq is determined from these face parts. At this point, the area of parts above the eyes of person 99, like first part P1, tends to fluctuate readily because of the influence of the hair, and thus these parts are not suitable for determining the center coordinate of estimation target region Sq from the standpoint of obtaining estimation target region Sq with a stable size. Meanwhile, the size of parts below the chin of person 99, like ninth part P9, tends to fluctuate readily because of the influence of clothing, and thus these parts are not suitable for determining the center coordinate of estimation target region Sq from the standpoint of obtaining estimation target region Sq with a stable size. Therefore, according to the present embodiment, the center coordinate is determined with first part P1 and ninth part P9 kept from being used in the determination of the center coordinate of estimation target region Sq, and estimation target region Sq is then determined with the determined center coordinate serving as its center.

Specifically, as can be seen in region generating image 12a linked by one of the solid-white arrows pointing to the right from face part region divided image 12 in the drawing, face part region C is determined that corresponds to the one or more face parts excluding any parts above the eye parts of person 99 and any parts below the chin part of person 99. Herein, face part region C is a region in which the face part regions shown within the dashed circle are merged together. Then, center of gravity Ca of face part region C is calculated. Thereafter, as can be seen in another region generating image 12a linked by the other solid-white arrow pointing to the right from region generating image 12a in the drawing, a rectangular region having its center at the coordinate of center of gravity Ca and having a size that includes therewithin a majority of the face is generated as estimation target region Sq. Estimation target region Sq is, for example, coordinate information corresponding to the original thermal image.

Referring back to FIG. 2, resolving power converter 103 converts, into a predetermined resolving power, a partial thermal image, within a thermal image, that corresponds to generated estimation target region Sq and a partial region probability image, within a region probability image, that corresponds to generated estimation target region Sq. In this example, the predetermined resolving power is set to the resolving power of 32×32 pixels, but the predetermined resolving power may be set to any resolving power. Resolving power converter 103 converts an input image to an image of the predetermined resolving power through at least one of a process of reducing a size of the input image or a process of interpolating the input image. As images are made to have a consistent resolving power by resolving power converter 103, the images input to outputter 104, that is, the images input to the trained body temperature estimation model are made to have a consistent resolving power. This configuration, as a result, renders it unnecessary, for example, to prepare separate body temperature estimation models for different resolving powers and use them according to the different resolving powers or to prepare a body temperature estimation model that can handle various resolving powers, and thus the configuration makes it possible to estimate the core body temperature with relatively less processing resources.

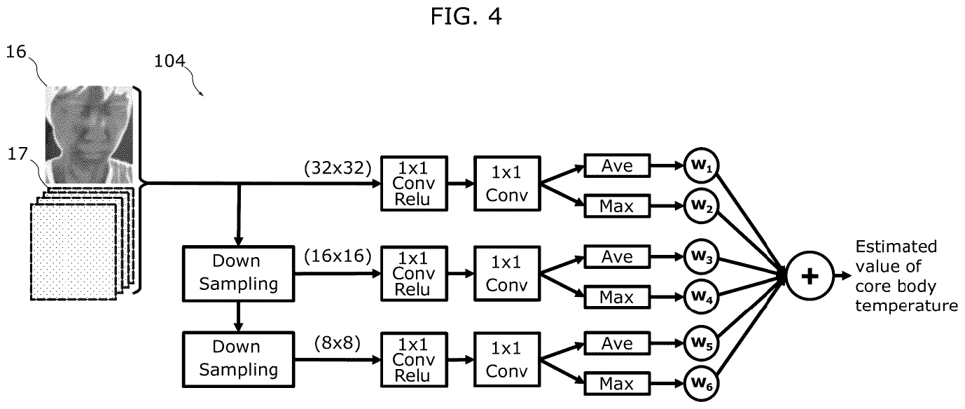

Outputter 104 inputs a partial thermal image converted to the predetermined resolving power and a partial region probability image converted to the predetermined resolving power into a trained body temperature estimation model, causes the body temperature estimation model to calculate an estimated value of the core body temperature of person 99, and outputs the calculated estimated value. FIG. 4 is a diagram for describing an operation of the outputter according to the embodiment.

As shown in FIG. 4, partial thermal image 16 converted to the predetermined resolving power and partial region probability image 17 converted to the predetermined resolving power are input to outputter 104. Outputter 104 inputs, into the body temperature estimation model, information of 32×32×pixel value corresponding to the temperature (1 channel) of partial thermal image 16 and information of 32×32×pixel value corresponding to the probability of each of the one or more face parts (e.g., 11 channels corresponding to 11 parts) of partial region probability image 17. In the body temperature estimation model, 12 types of linear sums of the 12 channels of each pixel are calculated at “1×1 Conv Relu”, and a nonlinear conversion of rounding up a value smaller than or equal to 0 to 0 is performed. 12×12 types of weighting factors corresponding to each channel at that point are determined through training. Then, 1 type of a linear sum between 12 types of linear sums calculated at each weighting factor at “1×1 Conv” is calculated. 1×12 types of weighting factors at that point are determined through training. As a result, an image having 32×32×1 pixel value corresponding to partial thermal image 16 converted to the predetermined resolving power and partial region probability image 17 converted to the predetermined resolving power is calculated. For this image, the mean value of all the pixels is calculated at “Ave”, and the calculated mean value is multiplied by weighting factor W₁. Weighting factor W₁ is determined through training. Furthermore, for the above image, the maximum value of all the pixels is calculated at “Max”, and the calculated maximum value is multiplied by weighting factor W₂. Weighting factor W₂ is determined through training.

Meanwhile, for the input image, the resolution of the image is reduced at “Down Sampling”, and an image of 16×16×12 channels is generated. In a similar manner, for the image of 16×16×12 channels, the resolution of the image is reduced at “Down Sampling”, and an image of 8×8×12 channels is generated. The process of reducing the resolution in this example may be any kind of process. For example, the process of reducing the resolution may be performed through a method in which the pixel value of one pixel is determined by the mean value of the pixel values of four pixels.

Then, in a similar manner, for the image of 16×16×12 channels whose resolution has been reduced and the image of 8×8×12 channels whose resolution has been reduced as well, the processes at “1×1 Conv Relu”, “1×1 Conv”, “Ave”, and “Max” are performed, and the results are multiplied by weighting factor W₃, weighting factor W₄, weighting factor W₅, and weighting factor W₆. Weighting factor W₃, weighting factor W₄, weighting factor W₅, and weighting factor W₆ are each determined through training.

Thereafter, the total value of the numerical values multiplied by the respective weighting factors is calculated, and thus an estimated value of the core body temperature is calculated. As described above, through the process of reducing the resolution, the broad relationship between the pixels can be taken into consideration in the estimation result of the core body temperature.

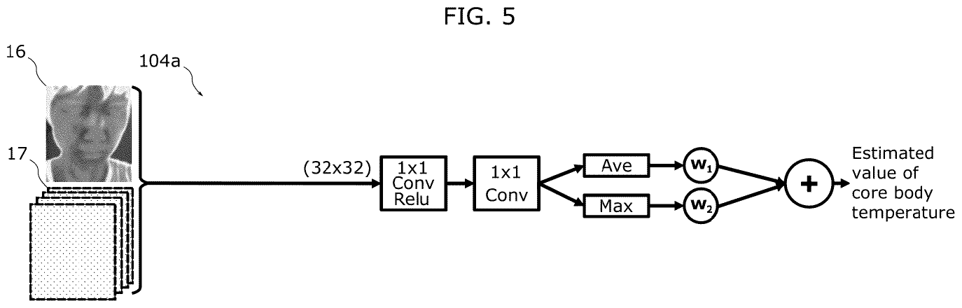

The body temperature estimation model described above is merely one example, and a different body temperature estimation model may also be used. FIG. 5 is a diagram for describing an operation of an outputter according to a different example of the embodiment. FIG. 5 shows outputter 104a including a different body temperature estimation model that can be adopted in place of the body temperature estimation model described above. For example, the body temperature estimation model of outputter 104a shown in FIG. 5 differs from the body temperature estimation model of outputter 104 described above in that the process of “Down Sampling” is omitted. In this case, although an estimation result having both a local relationship between the pixels and a broad relationship between the pixels cannot be obtained, the process of calculation for the estimation is greatly simplified, and high-speed core body temperature estimation device 100 of a simple configuration of a relatively low processing performance can be achieved. Herein, weighting factor W₁ and weighting factor W₂ in FIG. 5 are different from weighting factor W₁ and weighting factor W₂ of the body temperature estimation model of outputter 104 described above, and are determined through training.

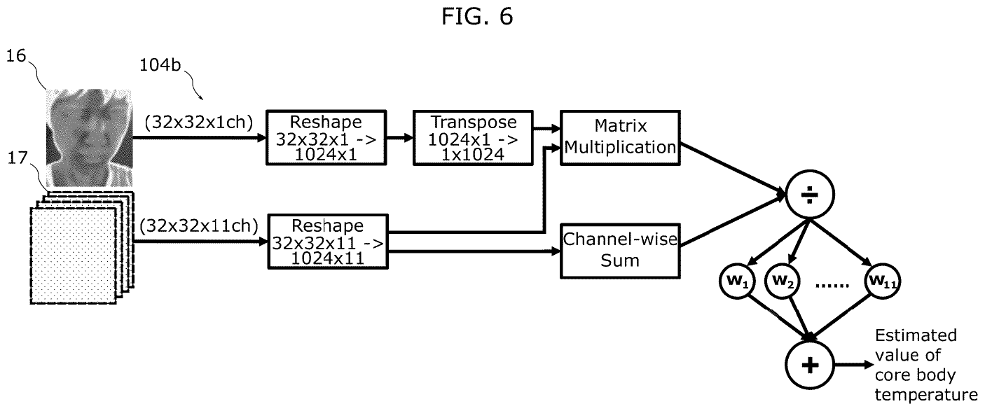

Meanwhile, FIG. 6 is a diagram for describing an operation of an outputter according to yet another different example of the embodiment. FIG. 6 shows outputter 104b including another body temperature estimation model that can be adopted in place of the body temperature estimation model described above. For example, the body temperature estimation model of outputter 104b shown in FIG. 6 calculates an all-pixel mean value in which a thermal image is weighted by the pixel value of each channel of partial region probability image 17, and calculates an estimated value of the core body temperature by obtaining a linear sum by multiplying each of the 11 mean values calculated for the 11 parts by corresponding weighting factor W₁ to weighting factor W₁₁. Specifically, as shown in FIG. 6, information of 1×1024 corresponding to partial thermal image 16 converted to the predetermined resolving power is generated at “Reshape 32×32×1->1024×1” and “Transpose 1024×1->1×1024”. In a similar manner, information of 1024×1 corresponding to partial region probability image 17 converted to the predetermined resolving power is generated at “Reshape 32×32×11->1024×11”. Then, the sum (a first sum) of the products of the temperature and the probability corresponding to each part is calculated by multiplying the two items of generated information at “Matrix Multiplication”, and the sum (a second sum) of the probability corresponding to the parts is calculated from the information of 1024×11 corresponding to partial region probability image 17 converted to the predetermined resolving power. Thereafter, the mean temperature of each of the 11 parts is calculated by dividing the first sum by the second sum to obtain the mean, and the calculated mean temperatures are multiplied by their corresponding weighting factor W₁ to weighting factor W₁₁ to obtain a linear sum. Thus, an estimated value of the core body temperature is calculated. Herein, weighting factor W₁ to weighting factor W₆ in FIG. 6 are different from weighting factor W₁ to weighting factor W₆ of the body temperature estimation model of outputter 104 described above, and are determined through training.

Aside from these other examples, any existing models for estimating a core body temperature may be used as a body temperature estimation model.

### Operation

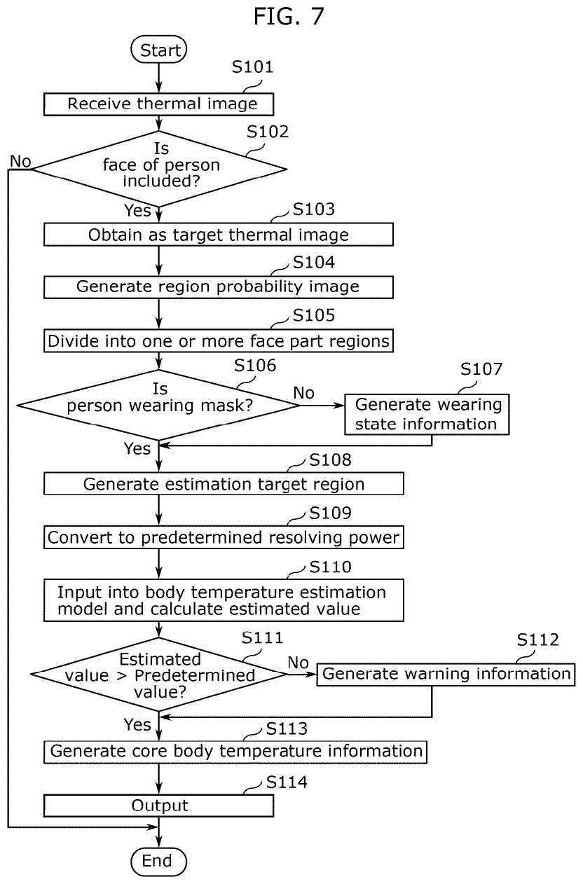

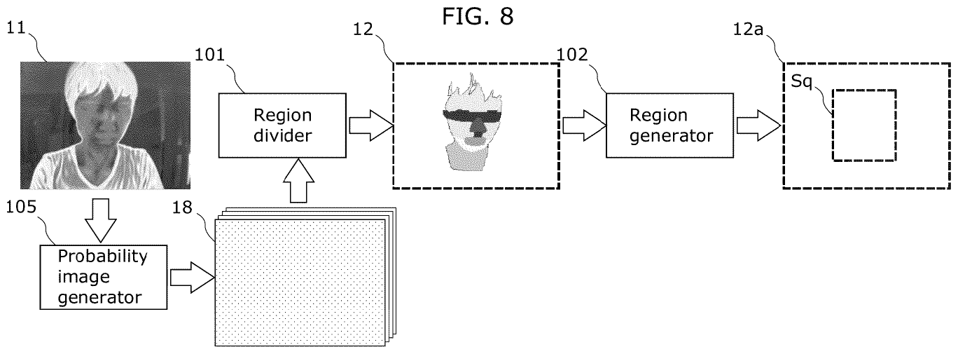

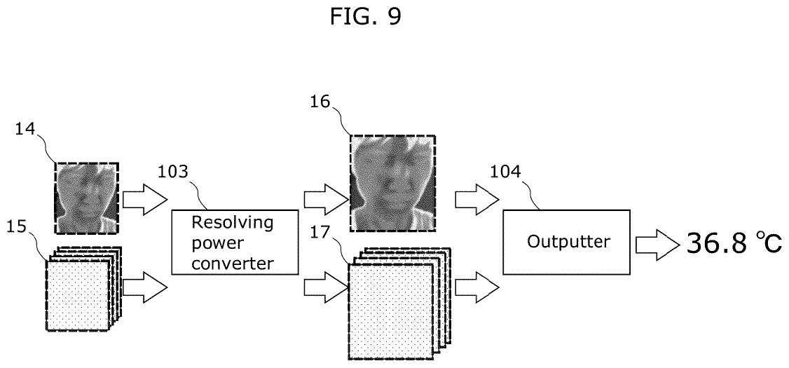

Next, an operation of core body temperature estimation device 100 and the core body temperature estimation system configured as described above will be described with reference to FIG. 7 to FIG. 9. FIG. 7 is a flowchart showing an operation of the core body temperature estimation device according to the embodiment. FIG. 8 is a first diagram for describing each image generated as the core body temperature estimation device according to the embodiment operates. FIG. 9 is a second diagram for describing each image generated as the core body temperature estimation device according to the embodiment operates.

As shown in FIG. 7 and FIG. 8, first, thermal image obtainer 106 receives a thermal image from imaging device 200 (step S101). In regard to the received thermal image, thermal image obtainer 106 determines whether this thermal image includes the face of person 99 (determination step S102). If thermal image obtainer 106 determines that the received thermal image does not include the face of the person (No at determination step S102), the process is terminated. Meanwhile, if thermal image obtainer 106 determines that the received thermal image includes the face of the person (Yes at determination step S102), thermal image obtainer 106 obtains this thermal image as target thermal image 11 (thermal image obtaining step S103).

Probability image generator 105 generates region probability image 18 from the obtained thermal image (probability image generating step S104).

Next, region divider 101 generates face part region divided image 12 by dividing the thermal image into one or more face part regions (region dividing step S105). At this point, outputter 104 determines whether person 99 is wearing a mask by determining whether there is a face part region, within face part region divided image 12, that corresponds to a face part having a mask on (step S106). If outputter 104 determines that person 99 is not wearing a mask (No at step S106), outputter 104 generates wearing state information that causes display device 300 to display a wearing state of a mask by person 99 (i.e., characters indicating, for example, that person 99 is wearing a mask or that person 99 is not wearing a mask) (step S107). Thereafter, the process proceeds to region generating step S108. Meanwhile, if outputter 104 determines that person 99 is wearing a mask (Yes at step S106), the process proceeds to region generating step S108 without outputter 104 performing any other process.

Region generator 102 generates estimation target region Sq with the use of face part region divided image 12 (region generating step S108). Thereafter, core body temperature estimation device 100 generates partial thermal image 14 corresponding to estimation target region Sq with the use of generated estimation target region Sq and target thermal image 11, and generates partial region probability image 15 corresponding to estimation target region Sq with the use of generated estimation target region Sq and region probability image 18.

Then, as shown in FIG. 7 and FIG. 9, through at least one of a process of reducing a size of the input image or a process of interpolating the input image, resolving power converter 103 generates partial thermal image 16 converted to the predetermined resolving power and partial region probability image 17 converted to the predetermined resolving power (resolving power conversion step S109). Outputter 104 inputs, into the body temperature estimation model, generated partial thermal image 16 converted to the predetermined resolving power and generated partial region probability image 17 converted to the predetermined resolving power, and causes the body temperature estimation model to calculate an estimated value of the core body temperature (step S110).

At this point, outputter 104 determines whether the estimated value of the core body temperature is anomalous by comparing the estimated value of the core body temperature against a predetermined value. To be more specific, outputter 104 determines whether the estimated value of the core body temperature is lower than the predetermined value (step S111). If outputter 104 determines that the estimated value of the core body temperature is higher than or equal to the predetermined value (No at step S111), outputter 104 generates warning information for issuing a warning by, for example but not limited to, sounding an alarm, displaying character information indicating a warning, or lighting a warning lamp (step S112). Thereafter, the process proceeds to step S113. Meanwhile, if outputter 104 determines that the estimated value of the core body temperature is lower than the predetermined value (Yes at step S111), the process proceeds to step S113 without outputter 104 performing any other process.

Outputter 104 generates core body temperature information that causes the display device to display in numerals the estimated value of the core body temperature that has been estimated (step S113). Then, outputter 104 outputs, from among the wearing state information, the warning information, and the core body temperature information, the information generated to display device 300 (output step S114). In this manner, the core body temperature estimated with high accuracy can be output even with a simple configuration.

Meanwhile, various cases are conceivable, including, for example, a case in which a person is wearing a mask, a case in which a person is not wearing a mask, a case in which a person is wearing eyeglasses, and a case in which a person is not wearing eyeglasses. Core body temperature estimation device 100 or core body temperature estimation system 500 according to the present embodiment always generates partial region probability image 17 corresponding to each face part covered, for example, by a mask or eyeglasses. Therefore, outputter 104 does not need to have or use different models for different cases in which the person is or is not wearing a mask or eyeglasses, and outputter 104 can be constituted by a single model. With this configuration, not only in cases in which a person is or is not wearing a mask or eyeglasses, but also in cases in which any face part is invisible because of the influence of the hair style or the orientation of the face, the core body temperature can be estimated with a highly robust single model.

Herein, the contents to be output to display device 300 are not limited to those described above. In the example described above, the wearing state of a mask serves as the wearing state information, but this is not a limiting example. The wearing state information may be served by information regarding the wearing state of eyeglasses or information regarding a state indicating whether the forehead is visible or invisible. Alternatively, the wearing state information does not need to be included. The same applies to the warning information and the core body temperature information as well, and the warning information or the core body temperature information does not need to be included in the output to display device 300. The estimated value of the core body temperature estimated by core body temperature estimation device 100 may be output to a device other than display device 300. For example, core body temperature estimation device 100 may be configured to output the estimated value of the core body temperature that has been estimated to a server or the like as numerical value information and to cause the server or the like to accumulate such numerical value information. In other words, outputter 104 in this case generates numerical value information of the estimated value of the core body temperature and outputs the generated numerical value information.

### EXAMPLES

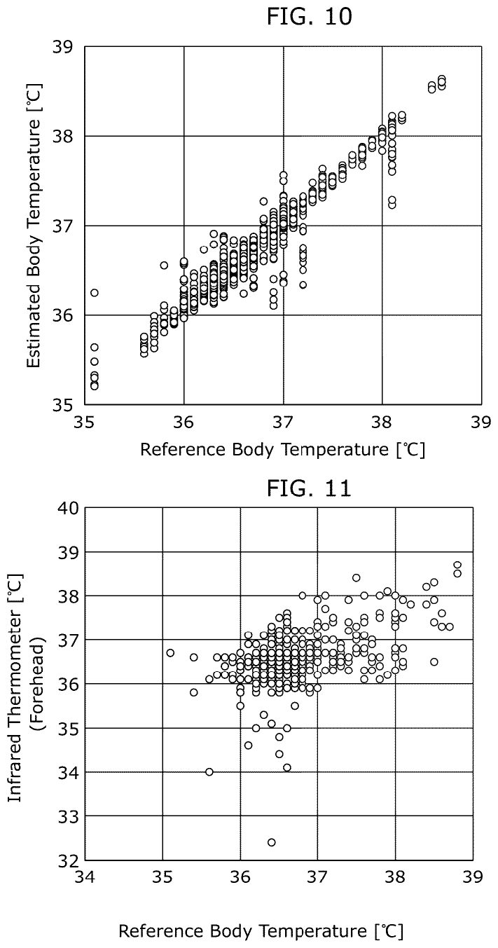

Some examples corresponding to the foregoing embodiment will now be described with reference to FIG. 10 and FIG. 11. FIG. 10 is a graph showing relationships between estimated values and correct values of core body temperatures according to an example. Meanwhile, FIG. 11 is a graph showing relationships between measured values and correct values of body temperatures measured by a body temperature measuring device according to a comparative example. In FIG. 10, the vertical axis represents the estimated value of the core body temperature output from the outputter, and the horizontal axis represents the measured value of the body temperature measured by a contact-type thermometer, which serves as the correct value close to the core body temperature. Meanwhile, in FIG. 11, the vertical axis represents the measured value of the temperature measured at the forehead with the use of a radiation thermometer, and the horizontal axis represents the measured value of the body temperature measured by a contact-type thermometer, which serves as the correct value close to the core body temperature.

As shown in FIG. 11, it can be seen that the variation in the correct value is large in the measurement of the body temperature with the use of a radiation thermometer. The RMSE calculated from this result is 0.610° C., and it can be seen that the correlation between the measured value and the correct value is very low. Meanwhile, as shown in FIG. 10, in the estimation of the core body temperature with the use of core body temperature estimation device 100 according to the foregoing embodiment, the correct value and the data are lined linearly, and it can be seen that the variation is small. The RMSE calculated from this result is 0.150° C., and it can be seen that the correlation between the measured value and the correct value is very high. Based on these results, it has been confirmed that the estimated value of the core body temperature according to the foregoing embodiment is accurate enough to withstand practical use.

### Other Embodiments

Thus far, the core body temperature estimation method and so forth according to the present disclosure have been described based on the foregoing embodiment, but the foregoing embodiment does not limit the present disclosure. For example, a process executed by a specific processor according to the foregoing embodiment may be executed by another processor. Furthermore, the order of a plurality of processes may be modified, or a plurality of processes may be executed in parallel. Although, in the example described according to the present disclosure, the core body temperature of one target person is estimated, the core body temperature of a plurality of persons may be estimated, and there is no limitation on the number of persons whose core body temperature is estimated.

The constituent elements according to the foregoing embodiment may each be implemented through the execution of a software program suitable for the corresponding constituent element. The constituent elements may each be implemented as a program executing unit, such as a central processing unit (CPU) or a processor, reads out a software program recorded on a recording medium, such as a hard disk or a semiconductor memory, and executes the software program.

Meanwhile, the constituent elements may each be implemented by hardware. For example, the constituent element may each be a circuit (or an integrated circuit). Such circuits may form a single circuit as a whole or may each be a separate circuit. Furthermore, these circuits may each be a general purpose circuit or a dedicated circuit.

General or specific aspects of the present invention may be implemented in the form of a system, an apparatus, a method, an integrated circuit, a computer program, or a computer readable recording medium, such as a CD-ROM. Furthermore, general or specific aspects of the present invention may be implemented through a desired combination of a system, an apparatus, a method, an integrated circuit, a computer program, and a recording medium.

For example, the present invention may be implemented in the form of an information processing method to be executed by a computer, may be implemented in the form of a program that causes a computer to execute such an information processing method, or may be implemented in the form of a non-transitory computer readable recording medium having such a program recorded thereon.

Aside from the above, an embodiment obtained by making various modifications that a person skilled in the art can conceive of to the foregoing embodiment or an embodiment achieved by combining, as desired, the constituent elements and the functions according to the foregoing embodiment within the scope that does not depart from the spirit of the present invention is also encompassed by the present invention.

### INDUSTRIAL APPLICABILITY

The present disclosure is widely useful as a technique that can replace measurement of human body temperatures.

### REFERENCE SIGNS LIST

11 target thermal image (thermal image)
12 face part region divided image
12a region generating image
14 partial thermal image
15 partial region probability image
16 partial thermal image converted to predetermined resolving power
17 partial region probability image converted to predetermined resolving power
18 region probability image
99 person
100 core body temperature estimation device
101 region divider
102 region generator
103 resolving power converter
104 outputter
105 probability image generator
106 thermal image obtainer
200 imaging device
300 display device
500 core body temperature estimation system
C face part region
Ca center of gravity
Sq estimation target region

1. A core body temperature estimation method of estimating a core body temperature of a person, the core body temperature estimation method comprising:
obtaining a target thermal image that shows a radiation temperature distribution of a target space including a face of the person;
generating a region probability image that shows a probability of each of a plurality of pixels in the target thermal image obtained corresponding to each of one or more face parts of the person;
dividing the target thermal image into one or more face part regions corresponding to the one or more face parts, based on the region probability image generated;
generating an estimation target region that includes, among the one or more face part regions, a region to be used to estimate the core body temperature of the person;
converting, of the target thermal image, a partial thermal image corresponding to the estimation target region generated to an image of a predetermined resolving power and converting, of the region probability image, a partial region probability image corresponding to the estimation target region generated to the predetermined resolving power; and
outputting an estimated value of the core body temperature of the person by inputting the partial thermal image converted to the predetermined resolving power and the partial region probability image converted to the predetermined resolving power into a body temperature estimation model trained and by causing the trained body temperature estimation model to calculate the estimated value of the core body temperature of the person.

2. The core body temperature estimation method according to claim 1, wherein
in the converting, an input image is converted to an image of the predetermined resolving power through at least one of a process of reducing a size of the input image or a process of interpolating the input image.

3. The core body temperature estimation method according to claim 1, wherein
the one or more face parts include at least one of a part that has on a mask serving as an accessory or a part that has on eyeglasses serving as an accessory.

4. The core body temperature estimation method according to claim 3, wherein
in the outputting, wearing state information that causes a display device to display a wearing state is output, the wearing state indicating whether the mask is worn.

5. The core body temperature estimation method according to claim 1, further comprising:
determining whether a thermal image includes the face of the person or does not include the face of the person, wherein
in the obtaining, a thermal image determined to include the face of the person is obtained as the target thermal image.

6. The core body temperature estimation method according to claim 1, wherein
in the generating of the estimation target region,
a center of gravity coordinate of a face part region is calculated, the face part region corresponding to one or more parts, among the one or more face parts, excluding a part above eye parts of the person and a part below a chin part of the person, and
a region having, as a center, the center of gravity coordinate calculated is generated as the estimation target region.

7. The core body temperature estimation method according to claim 1, wherein
in the outputting, core body temperature information that causes a display device to display the estimated value of the core body temperature is output.

8. The core body temperature estimation method according to claim 1, wherein
in the outputting, warning information for issuing a warning is output when the estimated value of the core body temperature is higher than or equal to a predetermined value.

9. The core body temperature estimation method according to claim 1, wherein
the outputting includes a process of calculating a mean value of all pixels in an image corresponding to the partial thermal image converted to the predetermined resolving power and the partial region probability image converted to the predetermined resolving power.

10. A non-transitory computer-readable recording medium having recorded thereon a program for causing a computer to execute the core body temperature estimation method according to claim 1.

11. A core body temperature estimation device that estimates a core body temperature of a person, the core body temperature estimation device comprising:
a thermal image obtainer that obtains a target thermal image that shows a radiation temperature distribution of a target space including a face of the person;
a probability image generator that generates a region probability image that shows a probability of each of a plurality of pixels in the target thermal image obtained corresponding to each of one or more face parts of the person;
a region divider that divides the target thermal image into one or more face part regions corresponding to the one or more face parts, based on the region probability image generated;
a region generator that generates an estimation target region that includes, among the one or more face part regions, a region to be used to estimate the core body temperature of the person;
a resolving power converter that converts, of the target thermal image, a partial thermal image corresponding to the estimation target region generated to a predetermined resolving power and converting, of the region probability image, a partial region probability image corresponding to the estimation target region generated to the predetermined resolving power; and
an outputter that outputs an estimated value of the core body temperature of the person by inputting the partial thermal image converted to the predetermined resolving power and the partial region probability image converted to the predetermined resolving power into a trained body temperature estimation model and by causing the trained body temperature estimation model to calculate the estimated value of the core body temperature of the person.

12. The core body temperature estimation device according to claim 11, wherein
the resolving power converter converts an input image to an image of the predetermined resolving power through at least one of a process of reducing a size of the input image or a process of interpolating the input image.
